(19) United States
(12) Patent Application Publication
(10) Pub. No.: US 2025/0378945 A1
(43) Pub. Date: Dec. 11, 2025

(54) SYSTEM AND METHOD FOR HEALTHCARE MANAGEMENT

(71) Applicants: Pulak Chakrabarti, GLENDORA, CA (US); Pallabi Chakrabarti, GLENDORA, CA (US)

(72) Inventors: Pulak Chakrabarti, GLENDORA, CA (US); Pallabi Chakrabarti, GLENDORA, CA (US)

(21) Appl. No.: 18/948,172

(22) Filed: Nov. 14, 2024

Related U.S. Application Data
(60) Provisional application No. 63/657,223, filed on Jun. 7, 2024.

(51) Int. Cl. G16H 40/67; G06F 40/20; G06Q 10/1093; G16H 10/60; G16H 15/00; G16H 80/00

(52) U.S. Cl. CPC G16H 40/67; G06F 40/20; G06Q 10/1093; G16H 10/60; G16H 15/00; G16H 80/00

(57) ABSTRACT

A system and method for managing the healthcare of a patient. The method includes rendering an interface on a user device for interacting with the system. Also, the method includes a digital assistant on the user device, wherein the digital assistant is configured to textually and verbally interact with the user. The digital assistant may be trained to recognize and understand the user's medical condition and medication compliance. The system can autonomously seek appointments and consultations with the patient.

### CROSS-REFERENCE TO RELATED APPLICATIONS

This application claims priority from a U.S. Provisional Patent Appl. No. 63/657,223, filed on Jun. 7, 2024, which is incorporated herein by reference in its entirety.

### FIELD OF INVENTION

The present invention relates to a system and method for healthcare management, more specifically, the present invention relates to artificial intelligence-based systems and methods for managing overall healthcare.

### BACKGROUND

Globally in healthcare management, the primary goal is to improve patient care while managing efficiency and cost-effectiveness. Both for patient care and improved outcomes of the treatment, frequent interactions of the doctor and the staff with a patient are essential. Moreover, extensive record-keeping is needed to monitor the medical condition of the user. Technology has greatly eased communication between caregivers and a patient. Also, many solutions for recordkeeping and analyzing the same have been reported. However, still, the overall process of patient care is largely complex, requires extensive manpower, and overall costs are significantly higher.

Moreover, the modern healthcare sector faces a multitude of challenges, including administrative burdens, data overload, and the need for personalized care.

A need is therefore appreciated for a system and method that can streamline the whole patient care process from needing medical consultation to post-visit patient care. There is a need for a system and method to overcome the limitations and problems in existing healthcare systems.

It is to be noted that the terms “health care” and “patient care” are interchangeably used herein and refer to providing services to a patient by healthcare personnel which includes doctors, medical staff, diagnostic centers, pharmacies, and the like. Similarly, the term caregiver refers to any person or an organization that renders its services directly or indirectly in managing the health of patients, and the term caregiver includes doctors, physicians, nurses, pharmacists, physiotherapists, psychologists, billing personnel, and the like persons related to the healthcare industry.

### SUMMARY OF THE INVENTION

The following presents a simplified summary of one or more embodiments of the present invention to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a system and method for artificial intelligence-based healthcare management.

Still, another object of the present invention is that the overall healthcare from patient contacting for a medical service and post-treatment can be managed.

Another object of the present invention is to make healthcare management cost-effective.

Still, another object of the present invention is that the response time in answering a patient can be improved.

Yet another object of the present invention is to address the challenges in the healthcare industry by automating routine tasks, analyzing vast amounts of data for insights, and enabling personalized patient interactions.

In one aspect, disclosed is a system for managing healthcare of a patient, the system comprising a processor and a memory, the system configured to implement a method including the steps of rendering, by the system, an interface on a user device, the interface configured to allow a user to interact with the system through the user device; rendering, by the system, a digital assistant on the user device, wherein the digital assistant is configured to textually and verbally interact with the user; training, the digital assistant about a user's medical condition and medication compliance; determining symptoms of a current medical condition of the user; automatically seeking an appointment for the current medical condition with a physician; rendering an interface on the user device for teleconsultation with the physician, wherein the digital assistant is configured to provide symptoms and medical records of the user to the physician; and updating the medical records of the user, by the system.

In one aspect, the method includes the steps of interpreting, using natural language processing (NLP), patient queries; providing evidence-based health information based on the patient queries; and preparing the user for an upcoming visit. The method further comprises receiving, by the system, one or more body parameters of the system, wherein the one or more body parameters comprise blood pressure measurements and glucose levels; and recording the one or more body parameters in a time series database. The method further comprises automating appointment settings and reminders.

In one aspect, the digital assistant is configured for capturing an audio conversation between the user and the physician; and processing the audio conversation into actionable data. The method further comprises incorporating the actionable data into the medical records.

The method further comprises rendering a dashboard screen on the user device, wherein the dashboard screen is configured to present a summary of the medical records.

In one aspect, disclosed is a method for managing healthcare of a patient, the method implemented within a system comprising a processor and a memory, the method including the steps of rendering, by the system, an interface on a user device, the interface configured to allow a user to interact with the system through the user device; rendering, by the system, a digital assistant on the user device, wherein the digital assistant is configured to textually and verbally interact with the user; training, the digital assistant about a user's medical condition and medication compliance; determining symptoms of a current medical condition of the user; automatically seeking an appointment for the current medical condition with a physician; rendering an interface on the user device for teleconsultation with the physician, wherein the digital assistant is configured to provide symptoms and medical records of the user to the physician; and updating the medical records of the user, by the system.

The method further comprises the steps of interpreting, using natural language processing (NLP), patient queries; providing evidence-based health information based on the patient queries; and preparing the user for an upcoming visit.

The method further comprises receiving, by the system, one or more body parameters of the system, wherein the one or more body parameters comprise blood pressure measurements and glucose levels; and recording the one or more body parameters in a time series database. The method further comprises automating appointment settings and reminders.

In one aspect, the digital assistant is configured for capturing an audio conversation between the user and the physician; and processing the audio conversation into actionable data. The method further comprises incorporating the actionable data into the medical records. The method further comprises rendering a dashboard screen on the user device, wherein the dashboard screen is configured to present a summary of the medical records.

### BRIEF DESCRIPTION OF DRAWINGS

The accompanying figures, which are incorporated herein, form part of the specification and illustrate embodiments of the present invention. Together with the description, the figures further explain the principles of the present invention and enable a person skilled in the relevant arts to make and use the invention.

FIG. 1 is a block diagram showing the environment of the system, according to an exemplary embodiment of the present invention.

FIG. 2 is a block diagram showing the architecture of the system, according to an exemplary embodiment of the present invention.

FIG. 3 is a flowchart illustrating a process implemented by the system, according to an exemplary embodiment of the present invention

FIG. 4 is a schematic diagram illustrating the functional architecture of the system, according to an exemplary embodiment of the present invention.

FIG. 5 shows an overview of the system, according to an exemplary embodiment of the present invention.

FIG. 6 shows a screen of the interface rendered on a user device, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

A healthcare management system and a method are disclosed to improve patient care while managing efficiency and cost-effectiveness. Disclosed systems and methods aim to transform the patient-doctor interaction by revolutionizing the global healthcare industry. Artificial intelligence-based healthcare systems may bring revolutionary transformation in doctor-patient interactions, appointment management, and record keeping. The disclosed system can integrate different aspects of healthcare using technologies, such as artificial intelligence, machine learning, image recognition algorithms, and natural language processing. The disclosed system can manage and augment every facet of the patient-doctor interaction, from pre-visit preparations to post-visit follow-up.

The disclosed system may implement a comprehensive solution, encompassing a range of functionalities that cater to various aspects of the healthcare process. These functionalities are integrated to create a seamless patient and healthcare provider experience.

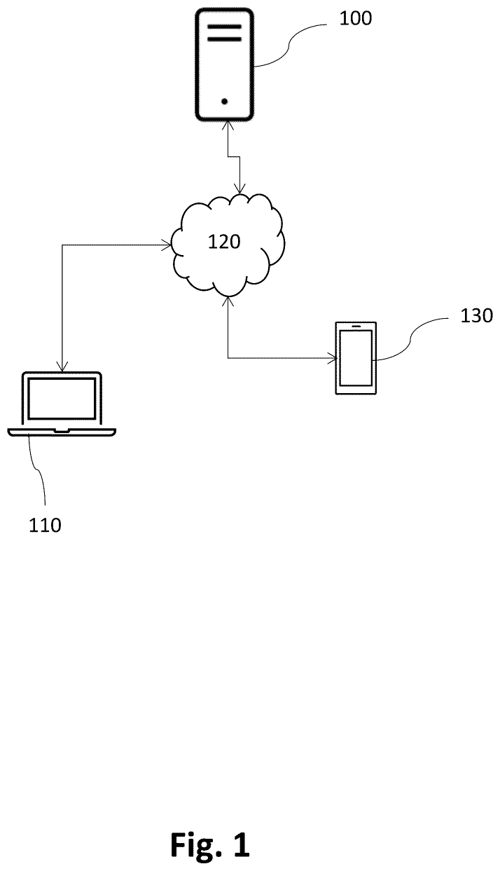

Referring to FIG. 1 which shows an environmental diagram of the disclosed system 100. The system 100 can connect to a patient device 110 and a caregiver device 130 through a network 120. The caregiver can be a doctor, medical staff, record keeper, clerical staff, and someone engaged in healthcare management. The patient and the caregiver are also referred to herein as a user. The term “user” as used herein, and throughout this disclosure, refers to an individual engaging a user device to interact with the system. Similarly, the term user device encompasses patient devices and caregiver devices.

The user device can be any computing device that includes a processor for processing instructions stored in memory. The user device can also include an input module for receiving input from the user. Such input can be in the form of a touch display, mouse, stylus, keyboard, touchpad, and the like. The user device may also include a display for presenting information to the user, for example, an LCD screen. The user device may also include a network circuitry for connecting to the network 120. Examples of the user device include a smartphone, a desktop computer, a laptop, a workstation, and the like.

The network can be a communication network known in the art which can be a wired network, a wireless network, or may include a combination of wired and wireless networks. Examples of communication networks may be a local area network (LAN), a wide area network (WAN), a wireless WAN, a wireless LAN (WLAN), a metropolitan area network (MAN), a wireless MAN network, a cellular data network, a cellular voice network, the Internet, etc. While, for illustration herein, FIG. 1 shows a single network connecting multiple user devices, it should be obvious to those reading this disclosure that different user devices can connect with the system through various networks, and the same user device can connect with the system through more than two networks. For example, a user device can connect to the system through a LAN and the Internet.

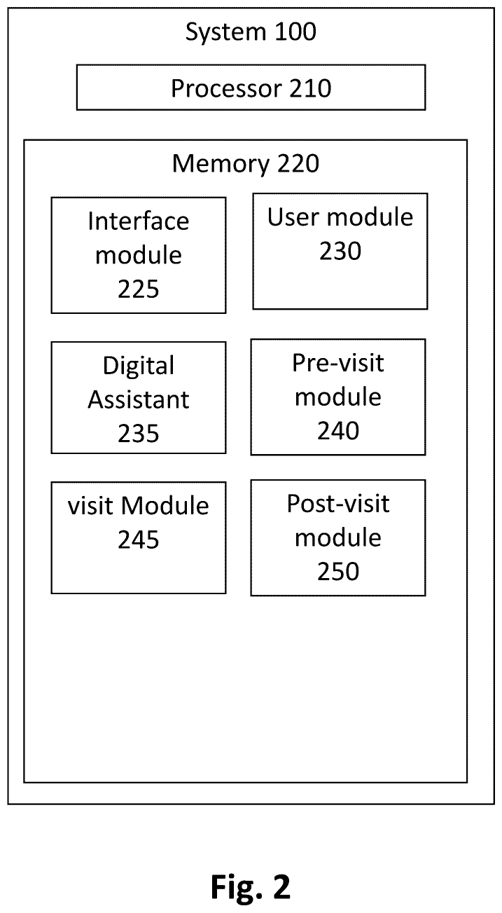

Referring to FIG. 2 which shows the architecture of system 100. The system 100 may include a processor 210 and a memory 220 operably coupled to the processor. The processor can be any logic circuitry that responds to, and processes instructions fetched from the memory. The memory may include one or more memory chips capable of storing data and allowing any storage location to be directly accessed by the processor. The memory can include modules according to the present invention for execution by the processor to perform one or more steps of the disclosed methodology.

The memory may include an interface module 225 which upon execution by the processor can render an interface on a user device allowing a user to interact with the disclosed system; a user module 230 which upon execution by the processor can allow an individual or an organization to register with the disclosed system; a digital assistant 235 which upon execution by the processor may verbally and textually interact with a user; a pre-visit module 240 which upon execution by the processor can handle pre-visit requirements of a patient, a visit module 245 which upon execution by the processor can assist in patient-doctor consultation and other activities in clinical settings; and a post-visit module 250 which upon execution by the processor can handle post-visit medical care of the patient.

The term module as used herein and throughout this disclosure refers to software, a program code, a set of rules or instructions, and the like in one or more computer-readable languages including graphics, which upon execution by the processor performs one or more steps of the disclosed methodology. Also, operations may be described as a sequential process, some of the operations may be performed in parallel, concurrently, and/or in a distributed environment, and with program code stored locally or remotely for access by single or multi-processor machines. In addition, in some implementations, the order of operations may be rearranged without departing from the spirit of the disclosed subject matter.

The system can be implemented in the form of servers, which include cloud servers. The servers can be placed in one location or geographically dispersed. Also, one or more steps of the disclosed methodology can be performed on one or more user devices without departing from the spirit of the disclosed subject matter.

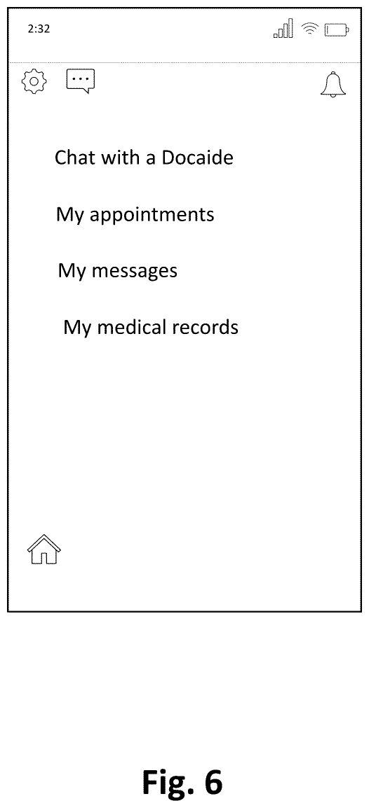

The interface provided by the interface module allows a user to interact with the disclosed system through a user device. The interface may include a series of screens, as shown in FIG. 6, which can provide information as well as receive information from the user and execute one or more steps of the disclosed methodology. The interface can be dynamic and allows switching between sections, screens, pages, and the like quickly and easily. The interface can be provided as an application software that can be installed on the user device.

The application software can be developed for Android™, iOS, and any other known operating platform for mobile devices. The application software can be made available through a distribution service provider, for example, Google Play™ operated and developed by Google, and the app store by Apple. In addition to the application software, a website-based interface can also be provided through the World Wide Web. The application software can also be provided for the desktop environment, such as Windows™, Linux, and macOS. The user interface may permit interaction with a user through the user device, wherein information can be presented within the user interface by system 100 and information can be received by system 100 from the user.

The user module may allow an individual willing to use the disclosed system to register with the system. The user module can receive basic information about the individual, such as name, contact details, email address, and the like. The user module can generate a profile for the user and store the same in a suitable database. The databases, including their structure and functioning, are known in the art. Also, the use of blockchain databases is well known and such databases may also be used. The present invention may use any suitable database without departing from the scope of the present invention. Also, the profile created by the user module can be later modified by the user. The user may have the option to edit entries or add more information to their profiles, and the user module can update the respective profile in the database.

The user module can generate login details to access the disclosed system securely. The login details may include at least a username and a password. The password can be an alphanumeric code, or biometric like a fingerprint, token, and the like. The user may have multiple login options, such as using an alphanumeric code or a fingerprint. Also, the use of multiple-factor authentication is within the scope of the present invention. The user can be provided with a login screen on the user device for accessing the disclosed system.

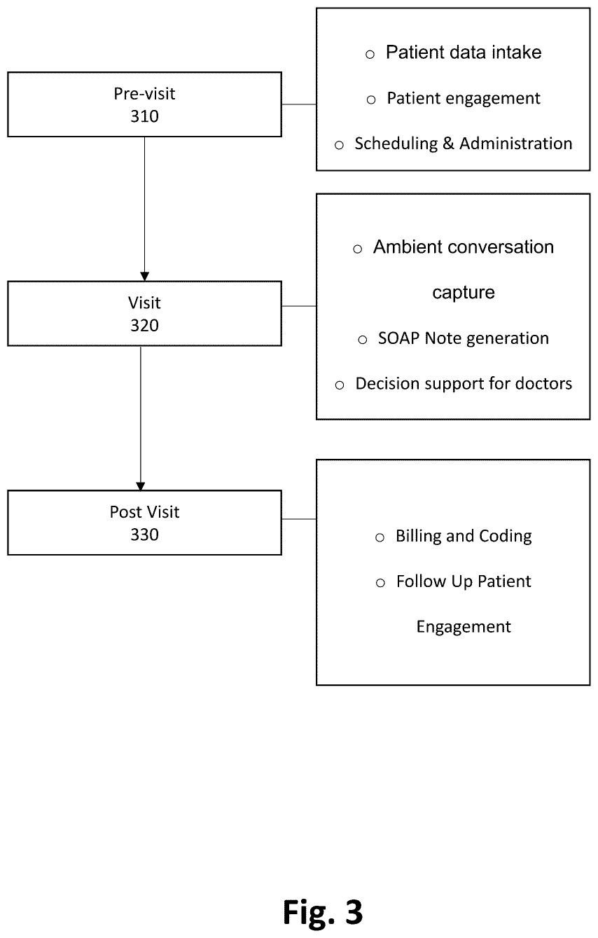

Referring to FIG. 3 which illustrates an overall process implemented by the system 100. The pre-visit module can handle the starting aspects of the healthcare process, at step 310. The pre-visit module may include the step of patient data intake. The pre-visit module may employ advanced data capture techniques to collect and organize patient information before a healthcare visit. This function streamlines the intake process by using artificial intelligence to guide patients through a series of questions and record their responses accurately. The pre-visit module may also allow for patient engagement, wherein the system can render a digital assistant on the user device. The digital assistant can engage with the user to gather health history and current symptoms. It uses natural language processing (NLP) to understand patient queries, provide evidence-based health information, and prepare the patient for their upcoming visit. The digital assistant can be adapted for each user wherein the digital assistant may have access to the user's medical records, past and present medical treatment, daily activities, medication, and the like. The digital assistant may act as a personal medical assistant to the user. Be it blood pressure measurements, daily glucose levels, medications, or any healthcare aspect, the digital assistant can track the same and make them available to the caregiver.

The pre-visit module can also handle the scheduling and administration aspects of healthcare. This may help both the caregiver in administration and the patients getting streamlined services. The system can automate the appointment setting and reminders. It integrates with the caregiver's calendars to optimize appointment slots and reduce wait times.

The visit module can also handle various aspects of healthcare management during the patient visit to the caregiver, as shown in step 320. The visit module may allow for ambient conversation recording functionality wherein the digital assistant can capture the conversation between the patient and doctor. It can recognize and record spoken language and process it into actionable data. This actionable data can be made a part of the patient's medical record and can be accessed as and when required. Clinical Documentation is one of the critical steps in healthcare management and is a labor-intensive process. The visit module can be trained to autonomously handle clinical documentation. The visit module, based on machine learning algorithms, can record the actionable data generated from conversations, any diagnosis or treatments prescribed, medicines dispensed, and the like into patient electronic health records (EHR), reducing manual data entry for healthcare providers. The system can connect with various EHR systems and can synchronize data across platforms, ensuring that patient records are up-to-date and accessible.

Although various aspects of medical data can be recorded, however, such data remains scattered. Determining any meaningful results from such data remains a challenge. The visit module can render dashboards on the user device that can summarize the data for the caregiver to review. The visits module can do analytics on the data to provide meaningful insights for the caregiver. This analysis can offer support to doctors by providing relevant medical information, potential diagnoses, and treatment options based on the patient's data and the latest medical research. For example, all the diagnostic information over a period of time, such as glucose levels, blood pressure levels, cholesterol levels, and the like parameters can be summarized on a single dashboard screen. Different colors and graphics can be used to show the trends and outliers. The dashboard screen may also allow the caregiver to navigate deep into the analysis. For example, a sudden rise in glucose levels for one or two days can be a result of missing medicine. The system can keep a record of both glucose levels and medication compliance by the patients.

Post-visit follow-up is a critical feature in improving the outcomes of treatment. The post-visit module can automate various aspects of post-visit follow-ups, as shown in step 330. Following the visit, the digital assistant continues to engage with patients to ensure they understand their care plan, medication instructions, and follow-up appointments. This feature aims to improve treatment adherence and patient satisfaction.

Accounting departments are common among healthcare service providers. The accounting department must be synchronized with all other departments to avoid any error in billing. The error may result in monetary losses for the patients or the service provider. The post-visit module can also automate this aspect of the healthcare process. The post-visit module can autonomously extract relevant information for billing and coding, thereby reducing errors and administrative time. It adapts to various coding standards to ensure accurate and efficient processing of claims and billing.

Various machine learning-based modules of the system may be built on self-improving algorithms that are learned from each interaction. The system refines its understanding and responses, becoming more effective over time. A feedback loop allows the system to adjust based on patient and healthcare provider inputs. This is crucial for tailoring the AI to the specific needs of each practice and patient population. Also, recognizing the sensitivity of health data, the disclosed system may incorporate robust security protocols to protect patient information, complying with standards, such as but not limited to HIPAA. In the healthcare industry, regulatory compliance is much more stringent. The disclosed system may keep up with changing healthcare regulations, ensuring that providers comply with all legal and ethical standards. These key functionalities collectively ensure that the system is not merely a tool but an integral component of the healthcare delivery system, facilitating enhanced patient care and operational efficiency.

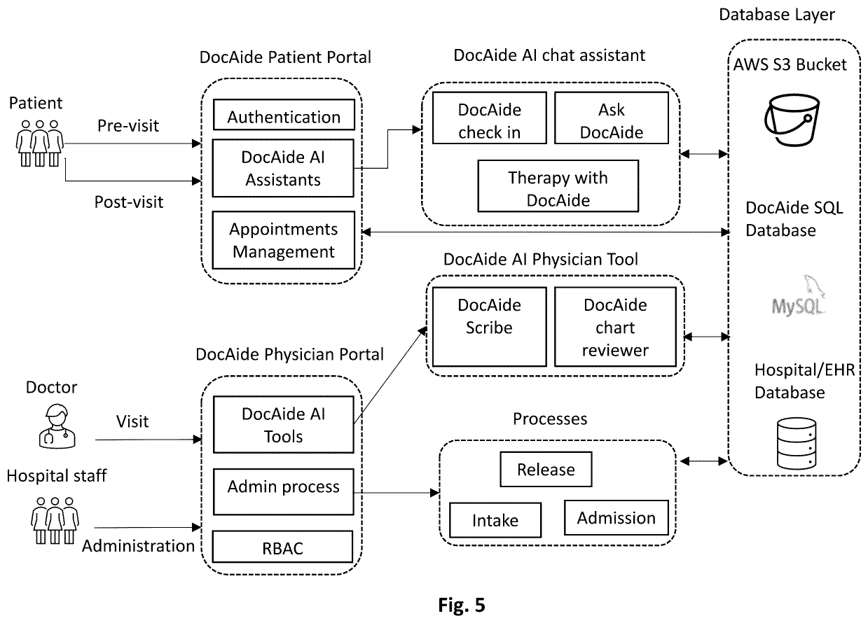

In certain implementations, the disclosed system may use a Retrieval-Augmented Generation (RAG) approach for dependable and accurate information delivery. This methodology enhances AI's responses, minimizing the likelihood of erroneous outputs, or “hallucinations,” common in the less advanced systems. FIG. 5 shows an overview of the system, detailing how each component of the system's infrastructure contributes to an enhanced patient-doctor interaction, aligning to revolutionize health and science technology through innovation.

In certain implementations, the machine learning models can use data for training. The data may come from a vast corpus of evidence-based healthcare data, ranging from medical records to scholarly articles, which forms the foundational knowledge base for the AI.

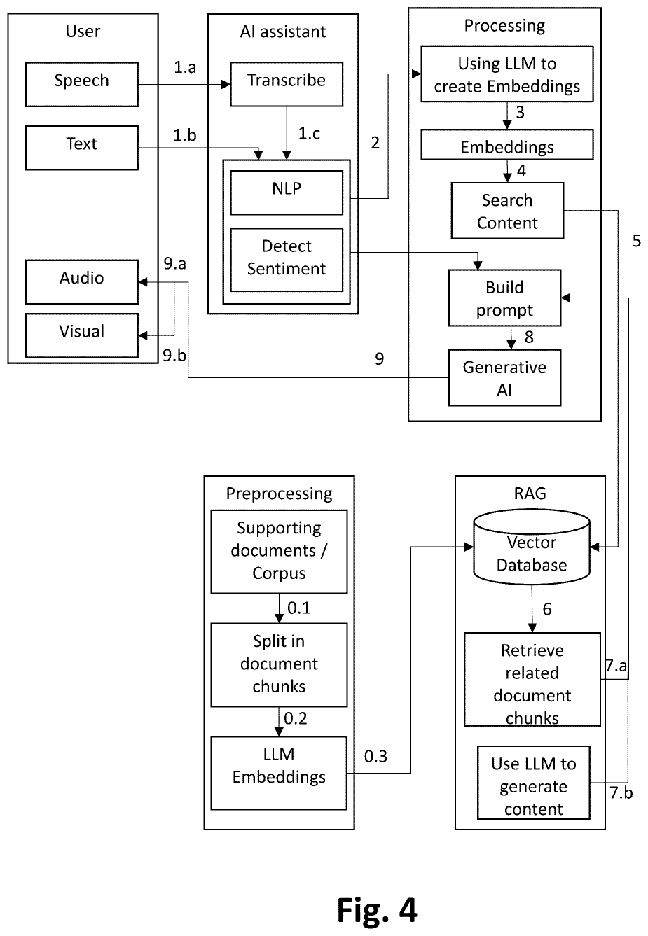

Referring to FIG. 4 which shows the technical approach architectural diagram:

### Preprocessing Stage:

(1) Supporting Documents/Corpus

The system begins by amassing a vast corpus of evidence-based healthcare data, ranging from medical records to scholarly articles, which forms the foundational knowledge base for the AI.
(2) Document Chunking

This corpus is then segmented into manageable chunks, allowing for more efficient processing and retrieval.
(3) LLM Embeddings

These chunks are processed to create Large Language Model (LLM) embeddings, which transform the text into a format that can be understood and utilized by AI algorithms.

AL Assistant Interface:
(1) Interaction with User: The AI avatar is the frontline interface for users, capable of understanding and processing both speech and text inputs (1.a, 1.b). It can also transcribe speech to text for further analysis (1.c).
(2) NLP and Sentiment Analysis—NLP is employed to comprehend and interpret the user's Language, including sentiment detection, crucial for personalizing patient interaction and care.

### Processing:

(1) Embeddings and Content Search—Using the LLM embeddings, the system may search its knowledge base to find relevant information.
(2) Building Prompts and Generative AI—Custom prompts are built to facilitate the Generative AI in creating responses or documentation that are contextually relevant and personalized to the patient's needs.

### RAG (Retrieval-Augmented Generation):

(1) Vector Database—A vector database is maintained for quick retrieval of information, enabling AI to provide real-time assistance and information access.
(2) Retrieval and Content Generation—The system may retrieve related document chunks (7.a) and employ the LLM to generate content such as health advice, administrative documentation, or support for clinical decision-making (7.b).
(3) Feedback Loop-Audio and Visual Feedback—The AI assistant is also capable of processing audio (9.a) and visual (9.b) feedback, enhancing its interactive capabilities and allowing for a more natural user experience.

In certain implementations, the AI Assistant Interface may be a frontline interface for users, capable of understanding and processing both speech and text inputs. It can also transcribe speech to text for further analysis. The AI assistant may also be capable of processing audio and visual feedback, enhancing its interactive capabilities and allowing for a more natural user experience.

Natural language programming may be employed to comprehend and interpret the user's Language, including sentiment detection, crucial for personalizing patient interaction and care. Using the LLM embedding, the system may search its knowledge base to find relevant information.

The system may be used by healthcare providers, clinics, and hospitals looking to improve efficiency and patient outcomes. With a focus on scalability and integration, the platform can serve small practices and Large healthcare systems alike.

The system may bring a range of benefits for patients, such as but not limited to improved engagement and education through conversational AI; Personalized healthcare experiences with sentiment-sensitive interactions; and better health outcomes through continuous post-visit engagement.

For healthcare providers, the system may offer reduced administrative burden through automated documentation; enhanced decision-making support with AI-generated insights; and increased efficiency with streamlined scheduling and billing processes.

In certain implementations, the AI assistant according to the present invention can utilize a Retrieval-Augmented Generation (RAG) based approach to conduct a step-by-step patient history and medical information intake, note down the reasons for the visit, and summarize the information for the doctor to review before the visit. Upon visiting, an AI-driven text and voice chat interface may be utilized for pre-visit patient check-ins. The AI-driven text and voice chat interface may further assist users with mental health concerns, utilizing a RAG-based approach to provide accurate medical information. Allows patients to make appointments and reach out to medical professionals for further help. For doctors, the system may allow them to record and transcribe patient sessions using advanced AI models. The AI assistant distinguishes speaker roles, summarizes key points, and generates SOAP clinical notes. The system may also allow doctors to upload and utilize custom templates.

The system may include a chart reviewer which may be a tool for doctors that utilizes an AI-driven chat interface that allows doctors to chat with an AI assistant and ask questions about the medical records of a patient. The AI assistant utilized RAG to extract key information and streamline the medical records management and the chart review process.

The system may also include user authentication and profile management for secure registration, login, and profile management for doctors, patients, and administrators. Another tool, Appointment Management, may be for scheduling, viewing, modifying, and canceling appointments with real-time doctor availability checks. Medical Records Management may allow for secure storage and management of patient medical records, including history, prescriptions, and diagnostics.

The system may utilize modular and extensible architecture: Designed to allow seamless addition of new features or updates without impacting existing functionality. The features serve as standalone components that can be integrated into the current Electronic Health Systems (EHRs) being utilized by hospitals and doctors. Interoperability and Integration: The system is designed to be integrated with existing hospital and EHR database systems like EPIC through secure API calls. The system will enable clinicians to access a comprehensive view of patient history and medical information transcending provider and geographical boundaries. The data exchange follows the industry standard format and guidelines. User-Centric Design: Provides an intuitive and responsive interface for both healthcare professionals and patients.

The interface according to the present invention may include actionable dashboards for patients and doctors and modular and interoperable UI for the AI chat assistants and AI doctor tools. Using AI Integration, the system may distinguish itself through its deep integration of cutting-edge AI technologies, which enhance the platform's capability to provide intelligent, personalized, and efficient healthcare services. The AI-driven functionalities in the system may be designed to transform the traditional healthcare experience by automating routine tasks, improving patient engagement, and delivering actionable insights to healthcare providers. HIPAA compliance and Advanced Security: the system may utilize industry-standard encryption, access controls, audit trails, and incident response measures, and data protection guidelines. The system provides a secure environment for healthcare providers and patients, ensuring trust and confidence in its platform.

Referring to FIG. 5 which shows the Technical Architecture Overview of system 100.

```
Technology Stack
    Frontend:
        React: For building a dynamic and responsive user interface.
        JavaScript: Manages client-side application logic.
        Tailwind CSS: Provides a modern, utility-first approach to styling the UI
  components.
    Backend:
        Python (Flask): A lightweight web framework that serves as the backend,
  handling HTTP requests, data processing, and business logic.
        MySQL: An SQL database used to store structured data related to users,
  appointments, and chats.
        OpenAI API: Powers AI-driven features such as chatbots, voice-to-text
  conversion, and intelligent response generation.
System Architecture
    Model-View-ViewModel (MVVM) Pattern:
        Separates the application into three layers:
          Model: Manages the core data and business rules.
          View: Represents the UI components seen by the user.
          ViewModel: Handles the data-binding logic between the Model and the
      View, ensuring a clean separation of concerns.
    Component-Based Design:
        Each major functionality (like user management, appointment scheduling, AI
  chat) is encapsulated in its own component or module, facilitating scalability and
  maintainability and interoperability.
    RESTful API Communication:
        The system relies on RESTful APIs for communication between the client and
  server, ensuring efficient data transfer in JSON format.
```

In certain implementations, the system may ensure the security and privacy of the data. Encryption of data-User credentials and sensitive data may be securely encrypted using industry-standard hashing algorithms. Patient data stored in the databases may be encrypted at rest using advanced encryption standards (AES-256). This protects sensitive information from unauthorized access, even if the storage media is compromised. Role-Based Access Control (RBAC)—The system may employ role-based access control to ensure that only authorized personnel can access or modify patient information. Each user is assigned specific roles (such as doctor, patient, or administrator), which dictate their access permissions to different parts of the platform. API Security—the system may integrate with external services like the OpenAI API using secure and authenticated connections. All API calls are encrypted and monitored to prevent unauthorized access or data breaches.

The disclosed system may be versatile by allowing integration with external service providers. The system may integrate into the clinical workflows, supporting various stages of patient care from initial check-ins to post-consultation follow-ups. This integration reduces manual tasks, minimizes errors, and enhances overall efficiency.

AI-Driven Patient Interaction: The system may utilize state-of-the-art AI models powered by the OpenAI API to create highly interactive and personalized patient experiences. The platform offers several AI-powered tools that assist in various stages of patient care.

AI-Powered Clinical Documentation: The system may scribe to offer real-time context-aware transcription and summarization. Automated identification of key information such as symptoms, diagnoses, and prescribed treatments. The context ensures that important medical details are accurately captured without redundancy. This reduces the administrative burden on doctors, allowing more time to focus on patient care.

Intelligent Data Analysis and Insights: The system Chart Reviewer utilizes advanced Machine Learning, Large Language Models (LLMs), and Natural Language Processing (NLP) techniques to analyze unstructured medical data and extract meaningful insights from large volumes of text, improving decision-making and patient care strategies.

Modular Design: Allows for easy extension and adaptation of the platform, enabling rapid development of new features.

Advanced Patient Interaction: Uses AI to provide more personalized and efficient patient care, setting it apart from traditional healthcare management solutions.

Example: A women patient named Meera, 55 years old and living in a remote location may be suffering for one month from imbalance, dizziness, falls, and neuropathic pain. Although being present in a remote location, she wants access to world-class medical expertise, commonly available in the city. Meera may register herself with the disclosed system through her smartphone. Upon providing the required information, the AI assistant can be assigned to Meera. The AI assistant may have access to the files and medical history including records of Meera. The AI assistant through textual, verbal, and visual interactions with the patient, can obtain the essential information about Meera's symptoms. The AI assistant can then determine the type of medical assistance needed for Meera based on the symptoms. For example, the patient will need a neurologist evaluation and the severity of the medical condition of the patient. Upon getting approval from the patient, the AI assistant can schedule an appointment for the patient with a renowned neurologist in the city. The system can organize all the medical data of the patient before visiting the doctor. The doctor may decide whether the patient should visit him, or a teleconsultation is possible. Whatever the mode of consultation, the doctor may have a good amount of the patient's medical history, and present the medical condition, maybe before meeting the patient. This saves the consultation time and also makes the discussion between the patient and the doctor more fruitful.

The system may also provide an interface for consultation between a caregiver and a patient. For example, a seamless interface for video visits between the doctor and the patient. The system may enable ambient content captured to listen to the conversation between the caregiver and the patient and generate consultation notes. Such consolation notes can be reviewed by the doctor and the doctor can modify them. The doctor may also generate a prescription through the interface and may also digitally sign it. The system through the interface may also suggest treatment options including diagnostic tests and medicines that may be prescribed. The system may prepare a follow-up calendar for follow-up and update the doctor's calendar accordingly. The system can then guide Meera for diagnostic tests as prescribed by the doctor.

The system can fetch the results of the medical tests through the server of the respective facility. The system can autonomously determine the medical condition of the patient using AI models. Also, the system can take the opinions of the doctor and other specialists before concluding. Meera may be urgently referred to a City Hospital for an in-person neurosurgical evaluation. The system can streamline the whole process for Meera saving her from frequent in-person doctor visits. This saves money and time for both consultation and travel. Any disease or disorder can be diagnosed early using the disclosed system and the patient can be guided to the best available treatment available. Patients are not limited to the medical and healthcare facilities available nearby. Also, all the features of telemedicine can be incorporated or associated with the disclosed system. Doctors through the disclosed system can be directly approached by the patients. The system may provide increased patient volume for the doctors. The doctors and other caregivers do not have to bother about appointments and billing, as the same can be handled by the disclosed system. Also, individual doctors and small clinics usually lack facilities for taking notes of consultations and updating the EMR of the patient. The system can handle this aspect of healthcare independent of the caregiver providing uniform and standardized practices. The system significantly reduces administrative burdens with AI-enabled scheduling, billing, and follow-ups. Better real-time support and reduced costs improve patient outcomes significantly. Also, the AI assistant can better present the case of the patient citing all the symptoms and facts. This helps the doctors to better understand the medical condition of the patient.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A system for managing healthcare of a patient, the system comprising a processor and a memory, the system configured to implement a method comprising the steps of:
rendering, by the system, an interface on a user device, the interface configured to allow a user to interact with the system through the user device;
rendering, by the system, a digital assistant on the user device, wherein the digital assistant is configured to textually and verbally interact with the user;
training, the digital assistant about a user's medical condition and medication compliance;
determining symptoms of a current medical condition of the user;
automatically seeking an appointment for the current medical condition with a physician;
rendering an interface on the user device for teleconsultation with the physician, wherein the digital assistant is configured to provide symptoms and medical records of the user to the physician; and
updating the medical records of the user, by the system.

2. The system of claim 1, wherein the method further comprises the steps of:
interpreting, using natural language processing (NLP), patient queries;
providing evidence-based health information based on the patient queries; and
preparing the user for an upcoming visit.

3. The system of claim 1, wherein the method further comprises:
receiving, by the system, one or more body parameters of the system, wherein the one or more body parameters comprise blood pressure measurements and glucose levels; and
recording the one or more body parameters in a time series database.

4. The system of claim 1, wherein the method further comprises:
automating appointment settings and reminders.

5. The system of claim 1, wherein the digital assistant is configured for:
capturing an audio conversation between the user and the physician; and
processing the audio conversation into actionable data.

6. The system of claim 5, wherein the method further comprises:
incorporating the actionable data into the medical records.

7. The system of claim 1, wherein the method further comprises:
rendering a dashboard screen on the user device, wherein the dashboard screen is configured to present a summary of the medical records.

8. A method for managing healthcare of a patient, the method implemented within a system comprising a processor and a memory, the method comprising:
rendering, by the system, an interface on a user device, the interface configured to allow a user to interact with the system through the user device;
rendering, by the system, a digital assistant on the user device, wherein the digital assistant is configured to textually and verbally interact with the user;
training, the digital assistant about a user's medical condition and medication compliance;
determining symptoms of a current medical condition of the user;
automatically seeking an appointment for the current medical condition with a physician;
rendering an interface on the user device for teleconsultation with the physician, wherein the digital assistant is configured to provide symptoms and medical records of the user to the physician; and
updating the medical records of the user, by the system.

9. The method of claim 8, wherein the method further comprises the steps of:
interpreting, using natural language processing (NLP), patient queries;
providing evidence-based health information based on the patient queries; and
preparing the user for an upcoming visit.

10. The method of claim 8, wherein the method further comprises:
receiving, by the system, one or more body parameters of the system, wherein the one or more body parameters comprise blood pressure measurements and glucose levels; and
recording the one or more body parameters in a time series database.

11. The method of claim 8, wherein the method further comprises:
automating appointment settings and reminders.

12. The method of claim 8, wherein the digital assistant is configured for:
capturing an audio conversation between the user and the physician; and
processing the audio conversation into actionable data.

13. The method of claim 12, wherein the method further comprises:
incorporating the actionable data into the medical records.

14. The method of claim 8, wherein the method further comprises:
rendering a dashboard screen on the user device, wherein the dashboard screen is configured to present a summary of the medical records.
